(19) United States
(12) Patent Application Publication
(10) Pub. No.: US 2011/0142837 A1
(43) Pub. Date: Jun. 16, 2011

(54) Method Of Treating Acute Respiratory Distress Syndrome

(75) Inventors: John D. Lambris, Bryn Mawr, PA (US); Konstantinos Ritis, Alexndroupolis (GR)

(73) Assignees: TRUSTEES OF THE UNIVERSITY OF PENNSYLVANIA, Philidelphia, PA (US); DEMOCRITUS UNIVESITY OF THRACE, Alexandroupolis (GR)

(21) Appl. No.: 12/669,695

(22) PCT Filed: Jul. 17, 2008
(86) PCT No.: PCT/US08/08730

§ 371 Date: Jul. 12, 2010

Related U.S. Application Data
(60) Provisional application No. 60/961,341, filed on Jul. 20, 2007.

(51) Int. Cl. A61K 39/395; A61K 38/08; A61K 38/10; A61K 38/02; A61P 11/00

(52) U.S. Cl. 424/135.1; 514/1.5; 424/133.1; 424/158.1

(57) ABSTRACT

Methods for treating and/or alleviating acute respiratory distress syndrome in an individual diagnosed with or at risk of developing acute respiratory distress syndrome are disclosed. The methods comprise administering a therapeutically effective amount of a complement inhibitor or a TNF-alpha inhibitor to the individual, wherein the complement inhibitor or the TNF-alpha inhibitor reduces or prevents tissue factor production in alveolar neutrophils, thereby treating the ARDS, or delaying or preventing onset of ARDS.

Pursuant to 35 U.S.C. §202(c), it is acknowledged that the United States government may have certain rights in the invention described herein, which was made in part with funds from the National Institutes of Health under Grant Number GM62134.

### FIELD OF THE INVENTION

The present invention relates to the field of pharmaceuticals and treatment of respiratory disorders. In particular, the invention provides methods for the treatment of acute respiratory distress syndrome and associated pulmonary conditions. The methods involve administration of a complement inhibitor or a TNF-alpha inhibitor, which reduces or prevents tissue factor production in alveolar neutrophils.

### BACKGROUND OF THE INVENTION

Various publications, including patents, published applications, technical articles and scholarly articles are cited throughout the specification. Each of these cited publications is incorporated by reference herein, in'its entirety. Full citations for publications not cited fully within the specification are set forth at the end of the specification.

The acute respiratory distress syndrome (ARDS) is a severe, and often life-threatening complication of several systemic disorders and direct injury to the lungs. It is associated with the high mortality rate (31-73%), primarily as a consequence of multiple organ failure and sepsis (1). The formation of fibrin-rich exudates (hyaline membranes) in the lumen of lung alveoli is a morphological hallmark of ARDS (3). Intra-alveolar fibrin deposition occurring as a result of damage to the capillary endothelium or the alveolar epithelium significantly contributes to the pathogenesis of ARDS by decreasing surfactant activity, which favors alveolar collapse, and by decreasing alveolar fluid clearance. The presence of fibrin in lung alveoli is accompanied by increased fibrin formation in the lung microvasculature, contributing to the loss of endothelial integrity and to thrombosis in the microcirculation. The injury to the pulmonary microcirculation resulting from inflammatory and thrombotic mechanisms likely contributes to the increase in dead space fraction, which may be an independent risk factor in the course of ARDS (4). The intra- and extravascular deposition of fibrin indicates an increased pro-coagulant activity of blood in the lung microvasculature and alveolar fluid.

Bronchoalveolar lavage fluid (BALF) from ARDS patients has been shown to have procoagulant activity (5-8) that is tissue factor (TF)-dependent (3, 5), and that is more profound during the first three days following the clinical diagnosis of ARDS (5). TF, which initiates coagulation in vivo, is present only in minimal amount in the circulating blood under physiological conditions in which tissue integrity is preserved (9). However, several inflammatory mediators, including complement anaphylatoxins and cytokines, up-regulate TF expression in circulating leukocytes and thereby increase the thrombogenic activity of the blood (9-11). Despite the extensive work that has been done to characterize the inflammation and fibrin deposition that occur in ARDS, the identity of the cellular elements and related inflammatory mediators that promote intra-alveolar coagulation are still elusive.

Experimental and clinical studies suggest a role of neutrophils in ARDS pathogenesis. Histologic analyses of autopsy specimens have revealed a pronounced accumulation of polymorphonuclear cells (PMN) in the injured pulmonary alveoli (12) and in the interstitial tissue of the lungs affected by ARDS (13, 14). In addition, PMN represent the predominating cell population in the BALF of ARDS patients (15). Animal studies have also demonstrated that intravenous injection of endotoxin results in the marked accumulation of neutrophils in the lung tissue (16, 17), suggesting that neutrophils are involved in the pathogenesis of ARDS.

Despite 40 years of research and advances in medical technology, ARDS-associated mortality remains high, and no pharmacological therapies improve effectively its clinical course. Four decades after the first description of ARDS and followed by multiple studies on this syndrome, the only approach that has been proven to decrease mortality is mechanical ventilation with a low tidal volume (2).

Thus, there is a need in the art for a method for treating ARDS. This invention addressed those needs.

### SUMMARY OF THE INVENTION

The invention provides a method for treating acute respiratory distress syndrome (ARDS) in an individual, or for treating an individual at risk for developing acute respiratory distress syndrome (ARDS). The method comprises administering a therapeutically effective amount of a complement inhibitor or a TNF-alpha inhibitor to the individual, wherein the complement inhibitor or the TNF-alpha inhibitor reduces or prevents tissue factor production in alveolar neutrophils, thereby treating the ARDS, or delaying or preventing onset of ARDS. The individual is preferably human. In embodiment, the tissue factor is alternatively spliced tissue factor isoform.

The complement inhibitor comprises one or more of a C5a inhibitor, a C5aR inhibitor, a C3 inhibitor, a Factor D inhibitor, a Factor B inhibitor, or a combination thereof. In one embodiment, the complement inhibitor is a C5a inhibitor or a C5aR inhibitor. In one aspect, the C5a inhibitor or C5aR inhibitor is acetyl-Phe-[Orn-Pro-D-cyclohexylalanine-Trp-Arg] (PMX-53), PMX-53 analogs, neutrazumab, TNX-558, eculizumab, pexelizumab or ARC1905, or any combination thereof.

In another embodiment, the complement inhibitor is a C3 inhibitor. In one aspect, the C3 inhibitor is compstatin, a compstatin analog, a compstatin peptidomimetic, a compstatin derivative, or any combinations thereof.

In certain embodiments, the TNF-alpha inhibitor is an antibody that binds and inhibit TNF-alpha activity. Alternatively, it is a soluble TNF-alpha receptor, or a tyrosine kinase inhibitor.

In another embodiment, the reducing or preventing of tissue factor production in alveolar neutrophils results in reduction or prevention of intra-alveolar fibrin deposition.

In yet another embodiment, the complement inhibitor is administered by pulmonary administration.

In the method of treating an individual at risk, in an embodiment, the individual exhibits one or more clinical conditions of acute lung injury (ALI), sepsis and the systemic inflammatory response syndrome (SIRS), severe traumatic injury, severe head injury, pulmonary contusion; severe pulmonary infection, aspirated vomited stomach contents, inflamed pancreas, severe trauma with shock, multiple transfusions, near drowning, smoke inhalation, or overdoses of narcotics, salicylates, tricyclic antidepressants or other sedatives.

Other respiratory conditions contemplated to be treated according to the method of the invention include idopathic interstitial pulmonary fibrosis, chronic obstructive pulmonary disease and asthma.

Other features and advantages of the invention will be understood from the drawings, detailed description and examples that follow.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a graph of clotting time as a function of various reaction conditions in a modified prothrombin time assay (mPT). H.N. is human neutrophils; PBS is phosphate buffered saline; H.S. is serum from healthy individuals; BAL is bronchoalveolar lavage fluid; anti-TF is anti-tissue factor antibody; ARDS serum is blood serum from subject with ARDS.

FIGS. 2A-2C are a series of images and graphs related to assessing TF expression in BAL fluid cells. FIG. 2A depicts two representative images of alkaline-phosphatase anti-alkaline-phosphatase (APAAP) immunostained peripheral blood neutrophils (panel I) and BAL fluid neutrophils (panel II) from an individual with ARDS. Neutrophil purity in BAL fluid—88%. FIG. 2B depicts the results of FACS analysis of TF expression in BAL fluid neutrophils compared with circulating neutrophils of an individual with ARDS. Neutrophil purity in BAL fluid—88%. FIG. 2C depicts two representative images of phagocytes immunostained for TF. Phagocytes are obtained from BAL fluid of an individual with ARDS. Multinucleated giant cells positive for TF expression are shown in the left panel (I). Alveolar macrophages are shown in the right panel (II). In both photos, neutrophils are also stained.

FIG. 3 is a graph depicting the relative quantification of TF and alternative spliced TF (asTF) in circulating PMNs and BALF PMN. Data were obtained in a 2^{-DDC}_{T} data analysis. Relative expression (indicated by bars) was based on the average DC_{T} values in Table 1.

FIGS. 4A-4E are a series of graphs and representative images depicting data related to TF-dependent pro-coagulant activity in the presence of C5a or TNF-alpha inhibition. FIG. 4A is a graph of graph of clotting time as a function of various reaction conditions in a modified prothrombin time assay (mPT). H.N. is human neutrophils; ARDS serum is blood serum from subject with ARDS; ARDS BAL is bronchoalveolar lavage fluid from an ARDS patient; C5aRA is an antagonist of the receptor for C5a; anti-TF is anti-tissue factor antibody. FIG. 4B is a series of representative images of neutrophils immunostained for TF expression under various conditions. Panel I depicts control healthy neutrophils (HN) incubated with 50 μl serum from an ARDS patient. Panel II depicts HN stimulated with 40 μl BAL fluid from an ARDS patient. Panel III depicts HN pretreated with C5aR antagonist (10 μM) stimulated with BAL fluid. Panel IV depicts HN incubated with BALF inhibited with anti-TNF-α (0.4 μg/μl). FIG. 4C depicts a graph of FACS analysis of HN identified by forward- and side-scatter characteristics, before and after stimulation of cells with BALF, as well as after C5aR and TNF-α inhibition studies. Shaded curve: control; Arrow 1: Control PMN+ARDS serum; Arrow 2: PMN BAL+stimulated; Arrow 3: PMN BAL stimulated+C5aRa; Arrow 4: PMN BAL stimulated+anti-TNF. FIG. 4D is an image of a Western blot analysis of TF expression. Lane I: Control HN (incubated with ARDS serum). Lane II: HN stimulated with 40 μl BALF. Lane III: HN stimulated with BALF pretreated with C5aR antagonist. Lane IV: HN incubated with BALF inhibited with anti-TNF-α. Lane V: TF expression from total cells extracts from BALF with a neutrophil purity of 84%. FIG. 4E is a graph showing the median and range of concentrations for C5a, TNF-alpha and IL-6 in BALF from ARDS patients (n=7).

FIG. 5 is a graph showing levels of cytokines in BALF. Medians and ranges of concentrations (pg/ml) for G-CSF, GROalpha, IFN-gamma, IL-1beta, IL-8, IFN-gamma inducible protein of 10 kD, MIP-1 beta, eotaxin, GM-CSF, IL-5, IL-10, MCP-3, MIP-1alpha, platelet-derived growth factor BB, and vascular endothelial growth factor detectable in BALF from ARDS patients are shown (n=7). Due to high differences in the levels of various cytokines in ARDS BALF, two (right and left) panels separated by a dashed line are presented for better clarity. Values on the left y axis refer to cytokines in the left panel, whereas values on the right y axis refer to cytokines in the right panel.

FIG. 6 is a graph of clotting time as a function of various reaction conditions in a modified prothrombin time assay (mPT). H.N. is human neutrophils; PBS is phosphate buffered saline; BAL is bronchoalveolar lavage fluid; and anti-TF is anti-tissue factor antibody. Comp a represents a group of 4 ARDS patients in which compstatin reduced the procoagulant activity. Comp b represents a group of 3 ARDS patients in which compstatin did not significantly reduce the procoagulant activity.

### DETAILED DESCRIPTION OF ILLUSTRATIVE EMBODIMENTS

Various aspects of the invention provide methods for alleviating and/or treating acute respiratory distress syndrome (ARDS) in an individual, or likewise treating an individual at risk of developing ARDS. Specifically, the method comprises administering a complement inhibitor or TNF-alpha inhibitor, as described in greater detail below.

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

Generally, the nomenclature used herein and the laboratory procedures in cell culture, molecular genetics, organic chemistry, and nucleic acid chemistry and hybridization are those well known and commonly employed in the art. Standard techniques are used for nucleic acid and peptide synthesis. The techniques and procedures are generally performed according to conventional methods in the art and various general references (e.g., Sambrook and Russell, 2001, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations would be understood by the skilled artisan as appropriate to practice the present invention.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, a “complement inhibitor” is a molecule that prevents or reduces activation and/or propagation of the complement cascade that results in the formation of C5a or C5a-mediated signaling through a C5a receptor. A complement inhibitor can operate on one or more of the complement pathways, i.e., classical, alternative or lectin pathway.

A “C3 inhibitor” is a molecule that prevents or reduces the cleavage of C3 into C3a and C3b.

A “C5a inhibitor” is a molecule that prevents or reduces the activity of C5a.

A a “C5aR inhibitor” is a molecule that prevents or reduces the binding of C5a to the C5a receptor.

A “Factor D inhibitor” is a molecule that prevents or reduces the activity of Factor D.

A “TNF-alpha inhibitor” is a molecule that prevents or reduces the activity of TNF-alpha.

The term “tissue factor” refers to both isoforms (full length and alternatively spliced) of tissue factor, unless the context specifies or suggests otherwise.

“Treating” refers to any indicia of success in the treatment or amelioration of the disease or condition. Treating can include, for example, reducing or alleviating the severity of one or more symptoms of the disease or condition, or it can include reducing the frequency with which symptoms of a disease, defect, disorder, or adverse condition, and the like, are experienced by a patient.

“Preventing” refers to the prevention of the disease or condition, e.g., ARDS, in the patient. For example, if a patient is treated with the methods of the present invention and is not later diagnosed with ARDS, it will be understood that ARDS has been prevented in that patient.

The term “treat or prevent” is sometimes used herein to refer to a method that results in some level of treatment or amelioration of the disease or condition, and contemplates a range of results directed to that end, including but not restricted to prevention of the condition entirely.

A “prophylactic” treatment is a treatment administered to a subject who does not exhibit signs of a disease or exhibits only early signs of the disease for the purpose of decreasing the risk of developing pathology associated with the disease. This term may be used interchangeably with the term “preventing,” with the understanding that such prophylactic treatment or “prevention” does not establish a requirement for complete prevention of a disease in the entirety of the treated population.

As used herein, a “therapeutically effective amount” is the amount of a composition sufficient to provide a beneficial effect to the individual to whom the composition is administered.

It is understood that any and all whole or partial integers between any ranges set forth herein are included herein.

The invention springs in part from the inventors' clear demonstration that the pro-coagulant properties of bronchoalveolar lavage (BAL) fluid in ARDS patients results from the induction of tissue factor. Specifically, the local production of functionally active tissue factor in intra-alveolar exudates underlies the pro-coagulant properties of BAL fluid. Furthermore, the inventors have demonstrated that neutrophils in BAL fluid do not merely acquire tissue factor from microparticles released by other cells but are in fact the main source of tissue factor in intra-alveolar fluid of ARDS patients. In addition, the inventors have discovered that local production of tissue factor by neutrophils in lung alveoli is dependent on C5a and TNF-α signaling. It is further demonstrated that blockade of C5a or TNF-α signaling inhibits TF expression in neutrophils.

Accordingly, the invention provides a method for alleviating and/or treating acute respiratory distress syndrome (ARDS) in an individual. The method comprises administering a therapeutically effective amount of a complement inhibitor or a TNF-alpha inhibitor to the individual. In particular, the complement inhibitor is selected for its ability to inhibit the activity of C5a. Advantageously, the method may be used as a prophylactic treatment. The method provides, for the first time, a pharmaceutical therapy that reduces the pro-coagulant properties of BAL fluid of ARDS patients. It is believed that that the reduction of the pro-coagulant properties will reduce or prevent the intra-alveolar fibrin deposition that results in formation of hyaline membranes. Hyaline membranes limit the area of active gas exchange, affect surfactant activity and contribute to vascular congestion (4). Thus, the method of the invention is designed to alleviate and/or treat one or more of these clinical aspects. Inhibition of the formation of hyaline membranes is contemplated to reduce or preclude the fibrosis of hyaline membranes, the development of which leads to irreversible changes that cannot be resolved. Accordingly, the method of the invention is contemplated to reduce or prevent ARDS pathophysiology.

The method of the invention may be practiced with any vertebrate subject to ARDS, including but not limited to, non-mammals and mammals. Mammals include humans, non-human primates, goats, sheep, horses, mice, rats and the like. Preferably, the method is practiced with a human.

The method may be practiced to alleviate and/or treat ARDS in an individual diagnosed with ARDS. The method may also be used as a prophylactic treatment in an individual at risk for developing ARDS. ARDS is a severe lung disease that can be caused by a variety of direct and indirect insults. ARDS can occur in adults and in children, including infants. A number of different clinical conditions are associated with the development of ARDS. Examples of such conditions include, but are not limited to: acute lung injury (ALI); sepsis and the systemic inflammatory response syndrome (SIRS); severe traumatic injury (especially multiple fractures), severe head injury, and pulmonary contusion; severe pulmonary infection, such as pneumonia; breathing in vomited stomach contents; inflamed pancreas; severe trauma with shock; multiple transfusions, particularly with pre-existing liver disease or coagulation abnormalities; patients who have nearly drowned; smoke inhalation; and overdoses of narcotics (e.g., heroin), salicylates, tricyclic antidepressants, and other sedatives. Patients diagnosed with any of these conditions are at risk for developing ARDS. However, the invention should not be construed as limited to use in subjects diagnosed with one or more of these conditions.

The invention further contemplates alleviation and/or treatment of other respiratory conditions by administering a therapeutically effective amount of a complement inhibitor to the individual. Other respiratory conditions include. but not limited to: idiopathic interstitial pulmonary fibrosis; chronic obstructive pulmonary disease (COPD) and asthma.

Inhibition of C5a Formation or Activity:

As demonstrated clearly herein, inhibition of C5a binding to C5aR is effective to reduce or prevent tissue factor expression in neutrophils present in alveolar fluid of ARDS patients, which in turn reduces the pro-coagulant activity of alveolar fluid. Reduction of the pro-coagulant activity of alveolar fluid is expected to reduce or prevent intravascular and/or alveolar fibrin deposition. Any inhibitor of C5a formation or activity may be used in the method of the invention. Inhibition of C5a formation or activity may be accomplished in a variety of ways. For instance, C5a activity may be inhibited directly by preventing or significantly reducing the binding of C5a to its receptor, C5aR. A number of C5aR inhibitors are known in the art. Acetyl-Phe-[Orn-Pro-D-cyclohexylalanine-Trp-Arg] (AcF[OPdChaWR]; PMX-53; Peptech) is a small cyclic hexapeptide that is a C5aR antagonist. Analogs of PMX-53 (e.g., PMX-201 and PMX-205) that also function as C5aR antagonists are also available (see for instance Proctor et al., 2006, Adv Exp Med Biol. 586:329-45 and U.S. Pat. Pub. No. 20060217530). Neutrazumab (G2 Therapies) binds to C5aR, thereby inhibiting binding of C5a to C5aR. Neutrazumab (G2 Therapies) binds to extracellular loops of C5aR and thereby inhibits the binding of C5a to C5aR. TNX-558 (Tanox) is an antibody that neutralized C5a by binding to C5a.

C5a activity may also be inhibited by reducing or preventing the formation of C5a. Thus, inhibition of any step in the complement cascade which contributes to the downstream formation of C5a is expected to be effective in practicing the method of the invention. Formation of C5a may be inhibited directly by inhibiting the cleavage of C5 by C5-convertase. Eculizumab (Alexion Pharmaceuticals, Cheshire, Conn.) is an anti-C5 antibody that binds to C5 and prevents its cleavage into C5a and C5b. Pexelizumab, an scFv fragment of Eculizumab, has the same activity. Similarly, ARC 1905 (Archemix), an anti-C5 aptamer, binds to and inhibits cleavage of C5, inhibiting the generation of C5b and C5a.

Formation of C5a may be inhibited indirectly by inhibiting the cleavage of C3, which yields C3b and C3a. Factor D is inhibited by diisopropyl fluorophosphate. TNX-234 (Tanox) binds to and inhibits Factor D. The skilled artisan is familiar with the steps of the complement cascade and is readily able to identify steps which contribute to the formation of C5a and thus may be targeted for inhibition. Therefore, complement inhibitors useful in the invention include, but are not limited to, C5a inhibitors, C3 inhibitors and Factor D inhibitors.

In a preferred embodiment, formation of C5a is reduced or prevented through the use of a C3 inhibitor. Preferably, the C3 inhibitor is compstatin or a compstatin analog, derivative, aptamer or peptidomimetic. Compstatin is a small molecular weight disulfide bonded cyclic peptide having the sequence Ile-Cys-Val-Val-Gln-Asp-Trp-Gly-His-His-Arg-Cys-Thr (SEQ ID NO. 1). Examples of compstatin analogs, derivatives and peptidomimetics are described in the art. See, for instance, U.S. Pat. No. 6,319,897, WO/1999/013899, WO/2004/026328, and Morikis et al (1999, “Design, Structure, Function and Application of Compstatin” in Bioactive Peptides in Drug Discovery and Design: Medical Aspects, Matsoukas et al., eds., IOS Press, Amsterdam NL).

An exemplary compstatin analog comprises a peptide having a sequence: Xaa1-Cys-Val-Xaa2-Gln-Asp-Trp-Gly-Xaa3-His-Arg-Cys-Xaa4 (SEQ ID NO. 2); wherein:

Xaa1 is Ile, Val, Leu, Ac-Ile, Ac-Val, Ac-Leu or a dipeptide comprising Gly-Ile;

Xaa2 is Trp or a peptidic or non-peptidic analog of Trp;

Xaa3 is His, Ala, Phe or Trp;

Xaa4 is L-Thr, D-Thr, Ile, Val, Gly, or a tripeptide comprising Thr-Ala-Asn, wherein a carboxy terminal —OH of any of the L-Thr, D-Thr, Ile, Val, Gly or Asn optionally is replaced by —NH₂; and the two Cys residues are joined by a disulfide bond. Xaa1 may be acetylated, for instance, Ac-Ile. Xaa2 may be a Trp analog comprising a substituted or unsubstituted aromatic ring component. Non-limiting examples include 2-napthylalanine, 1-naphthylalanine, 2-indanylglycine carboxylic acid, dihydrotryptophan or benzoylphenylalanine.

Another exemplary compstatin analog comprises a peptide having a sequence: Xaa1-Cys-Val-Xaa2-Gln-Asp-Xaa3-Gly-Xaa4-His-Arg-Cys-Xaa5 (SEQ ID NO. 3); wherein:

Xaa1 is Ile, Val, Leu, Ac-Ile, Ac-Val, Ac-Leu or a dipeptide comprising Gly-Ile;

Xaa2 is Trp or an analog of Trp, wherein the analog of Trp has increased hydrophobic character as compared with Trp, with the proviso that, if Xaa3 is Trp, Xaa2 is the analog of Trp;

Xaa3 is Trp or an analog of Trp comprising a chemical modification to its indole ring wherein the chemical modification increases the hydrogen bond potential of the indole ring;

Xaa4 is His, Ala, Phe or Trp;

Xaa5 is L-Thr, D-Thr, Ile, Val, Gly, a dipeptide comprising Thr-Asn or Thr-Ala, or a tripeptide comprising Thr-Ala-Asn, wherein a carboxy terminal —OH of any of the L-Thr, D-Thr, Ile, Val, Gly or Asn optionally is replaced by —NH₂; and the two Cys residues are joined by a disulfide bond. The analog of Trp of Xaa2 may be a halogenated tryptophan, such as 5-fluoro-1-tryptophan or 6-fluoro-1-tryptophan. The Trp analog at Xaa2 may comprise a lower alkoxy or lower alkyl substituent at the 5 position, e.g., 5-methoxytryptophan or 5-methyltryptophan. In other embodiments, the Trp analog at Xaa 2 comprises a lower alkyl or a lower alkenoyl substituent at the 1 position, with exemplary embodiments comprising 1-methyltryptophan or 1-formyltryptophan. In other embodiments, the analog of Trp of Xaa3 is a halogenated tryptophan such as 5-fluoro-1-tryptophan or 6-fluoro-1-tryptophan.

Other C3 inhibitors include vaccinia virus complement control protein (VCP) and antibodies that specifically bind C3 and prevent its cleavage. Anti-C3 antibodies useful in the present invention can be made by the skilled artisan using methods known in the art. See, for instance, Harlow, et al. (1988, In: Antibodies, A Laboratory Manual, Cold Spring Harbor, N.Y.), Tuszynski et al. (1988, Blood, 72:109-115), U.S. patent publication 2003/0224490, Queen et al. (U.S. Pat. No. 6,180,370), Wright et al., (1992, Critical Rev. in Immunol. 12(3,4):125-168), Gu et al. (1997, Thrombosis and Hematocyst 77(4):755-759) and Burton et al., (1994, Adv. Immunol. 57:191-280). Anti-C3 antibodies are also commercially available. Other C3 inhibitors include C3-binding and complement inhibitory secreted S. aureus extracellular fibrinogen-binding protein Efb (Lee et al., 2004, J. Biol. Chem. 279: 50710-50716) and the Efb homologous protein, Ehp (Hammel et al., 2007, J. Biol. Chem. 282: 30051-30061.

Inhibition of TNF-alpha Formation or Activity:

As demonstrated clearly herein, inhibition of TNF-alpha is also effective to reduce or prevent tissue factor expression in neutrophils present in alveolar fluid of ARDS patients, which in turn reduces the pro-coagulant activity of alveolar fluid. Any inhibitor of TNF-alpha formation or activity may be used in the method of the invention. TNF-alpha inhibitors include antibodies that bind and inhibit TNF-alpha activity, such as adalimumab and infliximab, soluble TNF-alpha receptors, such as etanercept, and tyrosine kinase inhibitors, such as tyrphostin AB556, tyrphostin AG126 and genistein. Preferably, the TNF-alpha inhibitor is an antibody. The skilled artisan can prepare anti-TNF-alpha antibodies using methods known in the art, as described elsewhere herein.

Pharmaceutical Compositions, Dosing and Administration:

The invention encompasses the use of pharmaceutical compositions comprising a complement inhibitor and/or a TNF-alpha inhibitor to practice the methods of the invention. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter develop in the art of pharmacology. In general, such preparatory methods include the step of bringing the active ingredient into association with a carrier or one or more other accessory ingredients, and then, if necessary or desirable, shaping or packaging the product into a desired single-or multi-does unit.

As used herein, the term “pharmaceutically-acceptable carrier” means a chemical composition with which a complement inhibitor may be combined and which, following the combination, can be used to administer the complement inhibitor to a mammal.

As used herein, the term “physiologically acceptable” ester or salt means an ester or salt form of the active ingredient which is compatible with any other ingredients of the pharmaceutical composition, which is not deleterious to the subject to which the composition is to be administered.

The pharmaceutical compositions useful for practicing the invention may be administered to deliver a dose of between 1 ng/kg/day and 100 mg/kg/day. In one embodiment, the invention envisions administration of a dose which results in a concentration of a complement or TNF-alpha inhibitor between 1 μM and 10 μM in an individual diagnosed with or at risk of developing ARDS. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The pharmaceutical composition may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, and the type and age of the animal.

A single complement inhibitor or TNF-alpha inhibitor may be administered or two or more different complement and/or TNF-alpha inhibitors may be administered in the practice of the method of the invention. In one embodiment of the invention, the method comprises administration of only a complement inhibitor or a TNF-alpha inhibitor. In other embodiments, other biologically active agents are administered in addition to the complement or TNF-alpha inhibitor in the method of the invention. Non-limiting examples of other biologically active agents useful in the invention include anti-inflammatories, such as glucocorticoids, vasodilators, such as inhaled nitric oxide, replacement pulmonary surfactants, antibiotics, analgesics, drugs to reduce anxiety, drugs to increase blood pressure and/or stimulate the heart and muscle relaxers. The method may be practiced with an individual to whom oxygen is administered, for instance, by face mask or by mechanical ventilation. The method may also be practiced with on-going treatment of the precipitating illness or injury.

Pharmaceutical compositions that are useful in the methods of the invention may be administered systemically in oral solid formulations, parenteral, ophthalmic, suppository, aerosol, topical or other similar formulations. In addition to a complement inhibitor or TNF-alpha inhibitor, such pharmaceutical compositions may contain pharmaceutically-acceptable carriers and other ingredients known to enhance and facilitate drug administration. Other formulations, such as nanoparticles, liposomes, resealed erythrocytes, and immunologically based systems may also be used to administer a complement inhibitor or TNF-alpha inhibitor according to the methods of the invention.

In view of the discovery that neutrophils are activated locally in the lung microvasculature or the alveoli in ARDS, a preferred route of administration of a pharmaceutical composition is pulmonary administration. Accordingly, a pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, including replacement pulmonary surfactant, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, in microbubbles for ultrasound-released delivery or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents including replacement pulmonary surfactants; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The invention is further described in detail by reference to the following examples. The examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods used in the following Examples are now described.

Description of patient population: All patients included in this study were recruited from the Intensive Care Unit of Academic Hospital in Alexandroupolis, Greece. The protocol was approved by the Institutional Review Board, and all procedures were in compliance with institutional guidelines. Informed consent was obtained from the closest relatives.

Seven (7) consecutive non-smoking patients (mean age: 55±17 years) participated in the study. All patients fulfilled the criteria for ARDS diagnosis; as proposed by the American-European Consensus Conference on ARDS (20): in particular, PaO₂/FiO₂<200; diffuse and bilateral alveolar infiltrates on chest radiograph; pulmonary capillary wedge pressure <18 mmHg with the absence of clinical evidence of left atrial hypertension; and an appropriate initiating (predisposing) condition. The clinical condition associated with the development of ARDS was sepsis in all seven patients.

All patients were mechanically ventilated according to the ARDSnet protocol (2). Ventilator setting adjustments were applied according to individual patient's requirements. General therapeutic measures (such as volume substitution and vasoactive or inotropic drugs) were administered as required and antibiotic therapy was guided by microbiological findings.

Flexible bronchoscopy and BAL collection were performed for diagnostic purposes, in order to obtain, isolate, and culture possible microorganisms. Thus, BAL collection timing was driven by clinical necessity and was not carried out at specific time points. However, all selected patients included in the study underwent bronchoscopy between the second and fourth day after ARDS diagnosis, since temporal changes in ARDS BAL fluid indicate that this time point is characterized by enhanced procoagulant activity (5).

Preparation of BALF-BLF collection, isolation of BALF cells and supernatants: BALF collection was performed as described previously (21). In brief, a fiberoptic bronchoscope (Olymbus BF 15, Tokyo, Tokyo Japan) was wedged in a segmental or subsegmental bronchus in areas of new or progressive densities. Six 20-milliliter (ml) aliquots of sterile 0.9% saline were infused and removed by gentle suction (recovery 20-30%). The first aspirated fluid reflected a bronchial sample and was used for microbiological screening, while the remaining aspirates were collected separately in sterile tubes. The BALF was then filtered through sterile gauze and immediately used in the study. About 15-25 ml of the filtered BALF was immediately centrifuged for 15 minutes at 800×g at 4° C. in order to isolate BALF supernatants for functional assays and stimulation/inhibition assays of healthy cells. The remaining pellet of BALF cells was washed in PBS, re-diluted in 1 ml PBS, and used for cell counts and cell viability assessment by trypan blue staining. Another portion of the cell pellet was immediately used for FACS analysis (in four of seven patients, the timing of the BALF collection was suitable for the FACS experiments, Table 1), and the remaining cells were smeared on glass slides for May-Grünwald-Giemsa staining and Alkaline-Phosphatase anti-Alkaline-Phosphatase (APAAP) immunostaining and then kept at −20° C. until use, or stored as pellets at −20° C. for later RNA extraction and western blot analysis. In the four cases in which the BALF amount was >20 ml and the total number of BALF cells was >8×10⁶ (Table 1), an additional neutrophil separation was also performed using Histopaque 1077 and 1119 double-gradient density centrifugation (Histopaque; Sigma-Aldrich Diagnostics, St Louis, Mo., USA) according to the manufacturer's instructions, in order to obtain more highly purified neutrophils (>95%) for higher accuracy in semi-quantitative real-time PCR analysis.

Isolation of peripheral blood neutrophils: Circulating leukocytes from all ARDS patients and from 4 healthy donors were immediately isolated from EDTA-treated peripheral blood. Peripheral blood neutrophils were separated by Histopaque-double gradient density centrifugation. The absolute number of neutrophils was adjusted to 2-3×10⁶ cells/ml in PBS. Approximately one-quarter (˜6-8×10⁵ cells in 250 μl PBS) was used for each stimulation or inhibition reaction. Cell purity (>98%), viability by trypan blue exclusion (>97%), and platelet contamination (<2 platelets/100 neutrophils) were assessed in all experiments. May-Grünwald-Giemsa staining did not reveal any platelets adhering to the neutrophils.

Reagents for inhibition studies: The following reagents were used in the experimental examples.

a) SB-290152, a selective non-peptide antagonist of the complement anaphylatoxin receptor C3aR, was used to analyze the involvement of C3aR signaling in the generation of neutrophil-derived tissue factor (22).

b) To block C5aR stimulation on neutrophils, a small cyclic hexapeptide (AcF-[OPdChaWR]; PMX-53) that acts as a selective C5aR antagonist was employed (23,24).

c) For TNF-alpha blocking assays, a recombinant anti-human antibody (HUMIRA, Abbott) was used.

d) IL-6 monoclonal antibody mouse-anti-human (MAB 206, R&D) was used for IL-6 inhibition studies.

Modified prothrombin time (mPT) assay: The supernatants from neutrophils that had been incubated with BALF supernatants or/and various agents were isolated by centrifugation at 1000×g for 10 minutes; the supernatants from these neutrophil preparations were checked a second time to confirm the absence of contaminating cells or platelets. The coagulation activities (TF/FVIIa binding activity) of the cell supernatants were determined using a modified prothrombin time (mPT) assay as previously described (18). After performing the “classic” prothrombin time (PT) test (100 microliter (μl) platelet poor plasma (PPP) plus 200 μl thromboplastin ISI 1.9 (Instrumentation Laboratory, Milan, Italy), the modified PT analysis was carried out. Namely, 120 μl of cell or BALF supernatant and 80 μl thromboplastin were added to 100 μl of PPP to measure the changes in PT. As a control for modified PT assays, 120 μl of PBS was used instead of cell supernatant, and the in vitro clotting time usually ranged from 31 seconds to 34 seconds.

To verify that the thromboplastic activity was due to TF, supernatants were incubated for 30 minutes with a specific mouse anti-human TF monoclonal antibody (Mab, No 4509, American Diagnostica, Greenwich, Conn., USA), 10 μg/ml, at room temperature. PT was then measured by the mPT method. Controls involved incubation with the same subclass and concentration of mouse anti-human antibodies.

Stimulation and inhibition studies: Neutrophils from healthy individuals were incubated for 120 minutes at 37° C. in PBS containing various stimulatory or inhibitory substances, in a total volume of 250 μl. Neutrophils were incubated with: 1) serum (50 μl) from healthy individuals; 2) serum (50 μl) from patients suffering from ARDS; 3) BAL fluid (40 μl) from ARDS patients; 4) BAL fluid from ARDS patients, together with normal neutrophils that had been pretreated for 30 minutes with the C5aR antagonist AcF-[OpdChaWR] (5 μM final concentration); 5) BAL fluid from patients with ARDS, together with normal neutrophils that had been preincubated for 30 minutes with the C3aR antagonist SB-290152, 10 μM final concentration); 6) BAL fluid from ARDS patients pretreated for 30 minutes with Humira® (Abbot Laboratories, Abbott Park, Ill.; anti-TNF monoclonal antibody, 0.4 μg/μl final concentration), together with normal neutrophils; or 7) BAL fluid from ARDS patients, together with normal neutrophils pretreated for 30 minutes with anti-IL-6 Mab (10 μg/ml final concentration). All the substances tested in these stimulation and inhibition studies that were effective in preliminary experiments were found to have dose-dependent activity; the doses chosen for further experiments were those that had generated peak levels of activity.

RNA extraction and relative quantitative real-time PCR analysis of TF mRNA synthesis: Total RNA was extracted from double-gradient purified BALF neutrophils, non-purified BALF neutrophils, and controls' and patients' peripheral blood neutrophils that had been collected at the same time with as the BALF. RNA was extracted using TRIZOL® reagent (Invitrogen, Carlsbad, Calif.) according to the manufacturer's instructions

Tissue factor isoform-specific real-time PCR was performed in order to quantify the relative expression levels in the two TF isoforms with coagulant properties, full-length TF (referred to hereafter as “TF”) and alternative spliced TF (asTF) (25), and to distinguish possible differences in TF among the various cell samples. Alternatively spliced TF lacks the transmembrane domain and is soluble. In each sample, TF, asTF, and GAPDH mRNA sequence-specific primers and probes for detection were applied as previously described (26). In order to amplify the two TF isoforms separately, the primers and probe for the detection of TF were designed to hybridize to exon 5, which asTF lacks. The asTF primers were designed to hybridize to exons 4 and 6, with the probe covering the unique exon 4/6 boundary, which is present in asTF (26).

TABLE 1
2^{-DDC}_{T} data analysis: Relative quantification of
TF and asTF in circulating and BALF neutrophils (PMN)

| | Average | Average | | | | |
|---|---|---|---|---|---|---|
| | C_{T} | C_{T} | | | | Difference |
| Sample | (target) | GAPDH | DC_{T} | DDC_{T} | 2-^{DDC}^{_{T}} | expression |
| 1. Blood PMN, TF | 30.48 | 21.7 | 8.78 ± 1.05 | 0 | 1 | |
| 2. BAL PMN, TF | 26.9 | 21.6 | 5.32 ± 1.04 | 3.46 ± 1.71 | 11.03 | x11.03 |
| | | | | | | (2 > 1)* |
| 3. Blood PMN, as TF | 35.41 | 19.37 | 16.04 ± 2.2 | 0 | 1 | |
| 4. BAL PMN, as TF | 29.7 | 18.3 | 11.44 ± 1.39 | −4.6 ± 1.99 | 24.25 | x24.25 |
| | | | | | | (4 > 3)** |

C_{T}: Threshold value. DC_{T} = Average target C_{T} − Average GAPDH C_{T}.
DDC_{T} (for the same target gene) = Average DC_{T} of BAL PMN − Average DC_{T} of circulating PMN.
DDC_{T} = 0 when unstimulated cells are used as calibrator.
2^{-DDC}_{T} = Normalized target gene amount (TF or asTF) relative to circulating PMN target gene amount.
Numbers in italics indicate the fold increase in a target gene in BAL PMN when compared to the same type of circulating PMN.
*p < 0.002
**p < 0.008.

Two microliters of each cDNA, prepared from 0.5 μg total RNA using SuperScript III reverse trancriptase according to the manufacturer's protocol (Invitrogen Life Technologies), was added to a final PCR reaction volume of 25 μl that contained 12.5 μl TaqMan® Universal Master Mix (Applied Biosystems, Foster City, Calif.), 0.5 μl (10 μM) of each primer (Biolegio, Nijmegen, the Netherlands), 0.5 μl (10 μM) probe, and 11.5 μl H₂0. Real-time PCR was performed using the MiniOpticon Real-Time Detection System (Biorad) under the following conditions: 50° C. for 2 minutes, 95° C. for 10 minutes, 40 cycles of 95° C. for 15 seconds, and 60° C. for 1 minute. Standards for each target were generated by real-time PCR, and serial dilutions of each standard were used to construct the respective standard curves.

The 2^{-DDC}_{T} method (27) was used for quantification of the target genes (TF and asTF). In brief, the amount of target gene (TF or asTF) in the BAL-purified neutrophils was normalized to that of the patients' peripheral blood neutrophils (2^{-DDC}_{T}, Table 1). The same method was used for non-purified neutrophils from BALF. The averages of the threshold values (C_{T}), as well as the DC_{T} and DDC_{T} values for the 2-^{DDC}_{T} equation, are shown in Table 1. Given the limited sample number, a non-parametric test (the paired sign test) was used to compare data within each group. The level of statistical significance was set to p<0.05.

Alkaline-Phosphatase anti-Alkaline-Phosphatase (APAAP) Immunostaining: Smears of cells (BALF pellet and peripheral blood-isolated neutrophils, before and after stimulation and/or inhibition studies) were stored at −20° C. prior to use. Immunocytochemical staining for TF was performed using the APAAP method, as previously described (28). An IgG1 mouse anti-human TF Mab recognizing amino acids 1-25 (No 4509, American Diagnostica, Greenwich, Conn., USA) was used in this study. The same procedure was followed for CD11b staining, using an anti-CD11b (Mac-1) Mab (M0741, DakoCytomation, Carpenteria, Calif., USA); an IgG1 anti-CD19 Mab (M0740 DakoCytomation, Carpenteria, Calif., USA) was used as a negative control.

FACS analysis of BALF cells and neutrophils isolated from peripheral blood: Approximately 5×10⁵ cells originating from BALF pellets, re-diluted in 1 ml PBS (see preparation of BALF, Materials and Methods), and the same number of neutrophils isolated from peripheral blood were fixed and permeabilized for intracellular staining using the FIX & PERM system (Caltag Laboratories, Austria) according to the manufacturer's instructions. Indirect FITC labelling of cells was used, since this experimental protocol yielded more efficient staining than did another approach using FITC-conjugated TF Mab. Cells were stained with 1.8 μl (1.0 μg/μl stock solution) of the mouse anti-human TF Mab (4509) for 5 hours at 4° C., and the tubes were shaken every 30 minutes for optimum efficiency. The cells were then washed and incubated for 30 minutes at 4° C. with FITC-labeled goat anti-mouse secondary antibody (555988 BD Pharmingen). FITC-labeled mouse IgG1 (BD Biosciences) was used as an isotype control. Flow cytometry was performed using a FACScan with CellQuest software (Becton Dickinson, Mountain View, Calif.), and neutrophils were identified by forward- and side-scatter characteristics.

Western blot analysis: Cell lysates were prepared from ˜2×10⁶ cells using a lysis buffer containing 1% Triton-X in 150 mM NaCl/20 mM Hepes, pH 7.5, with protease inhibitors (Complete Protease Inhibitor Tablets, Roche) at −20° C. Western blot analysis was performed as previously described (18).

Luminex assay: Concentrations of growth factors, cytokines, and inflammatory mediators in BALF from ARDS patients was measured using a Procarta cytokine profiling kit, according to the manufacturer's instructions (Panomics). After incubation with antibody-conjugated beads, detection antibodies and streptavadin-PE complexes, samples were run on Bio-Plex instruments (Bio-Rad). Levels of the following growth factors, cytokines, and inflammatory mediators were evaluated: eotaxin, fibroblast growth factor-basic, G-CSF, CM-CSF, growth related gene product (GRO-alpha), IFN-gamma, IL-1 alpha, IL-1 Beta, IL-2, IL-4, IL-5, IL-6, IL-7, IL08, IL-10, IL-12 (p40 and p70), IL-13, IL-17, IFN-gamma inducible protein of 10 kD, leptin, MCP-3, MIP-1 alpha, MIP-1 beta, nerve growth factor, platelet derived growth factor BB, RANTES, TNF-alpha, TNF-beta, and vascular endothelial growth factor.

C5a detection by ELISA: C5a levels in ARDS-BALFs were measured by ELISA using customized compositions and methods that detected only the C5a fragment of C5. Briefly, plates were coated with anti-C5a mAb (R&D Systems), then recombinant C5a (Sigma-Aldrich), which was used to generate a standard curve, and BALF samples were serially diluted on those plates. Polyclonal Abs against N- and C-terminal peptides of C5a (each 21 residues in length) were used to detect bound C5a. Polyclonal anti-rabbit HRP-conjugated IgG (Bio-Rad) was applied to detect C5a with Abs bound to Abs.

Statistical analysis: All data are expressed as means±SD. Statistical analyses were performed using Student's t-test in order to compare differences in means. The level of significance was set to p<0.05. Data were processed using the STATISTICA, version 5.0 (Statsoft®, Tulsa, Okla.), statistical program for Windows.

### EXPERIMENTAL EXAMPLE 1

### BALF from ARDS Patients has Strong Pro-Coagulant Activity that is TF-Dependent+

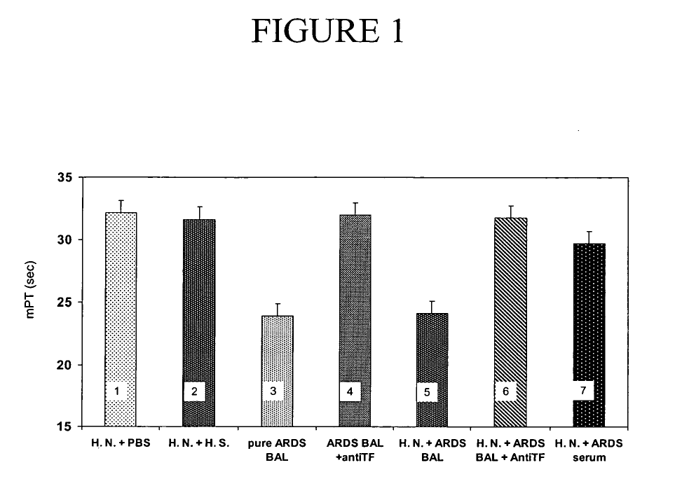

To evaluate the pro-coagulant activity of BALF from ARDS patients, the mPTs of BALF supernatants obtained after centrifugation were measured. Preliminary experiments showed that the pro-coagulant activity of BALF was dose-dependent, reaching maximal levels when 150 μl of BALF was used for the assay; 40 μl of BALF had no effect on the mPTs, when compared to control values. For further experiments, a dose of 120 μl of BALF was used, since this amount of fluid induced significant shortening (p<0.001) of the mPT (23.91±1.30 sec; FIG. 1, bar 3) when compared to mPT of supernatants from neutrophils incubated with serum of healthy individuals (31.62±0.45 sec; FIG. 1, bar 2), or PBS alone (32.11±0.58 sec; FIG. 1, bar 1).

These assays further demonstrated that the pro-coagulant properties of BALF are dependent on the presence of functionally active TF, since pre-incubation of BALF supernatants with neutralizing anti-TF Mab completely abolished the pro-coagulant activity (31.95±0.74 sec; FIG. 1, bar 4).

### EXPERIMENTAL EXAMPLE 2

### Neutrophils Acccumulating in the Lumen of Pulmonary Alveoli in ARDS Patients are a Major Source of TF

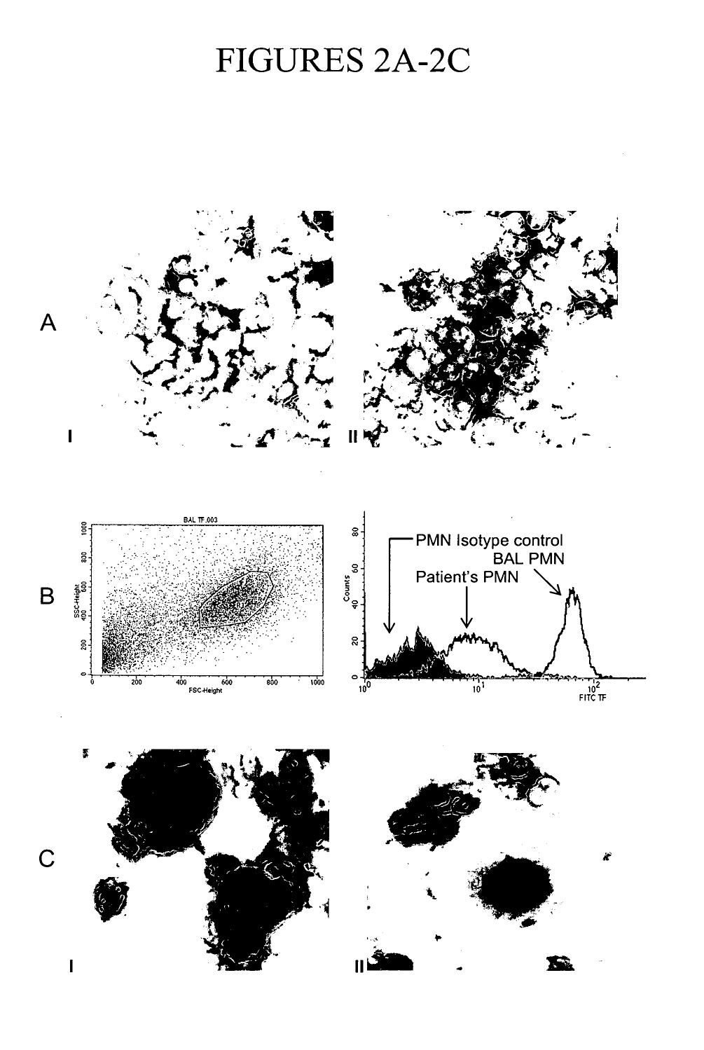

Neutrophils constitute the major cellular population in the BALF from ARDS patients (Table 2). To test the hypothesis that neutrophils express TF within the alveoli of ARDS-affected lungs, the expression of TF in various cell types from the BALF of ARDS patients was characterized. Immunohistochemical staining of smears prepared from this fluid showed that >85% of neutrophils expressed TF in all seven analyzed samples, whereas no expression or only weak staining was observed on peripheral blood neutrophils from the same patients (FIG. 2A). Multinucleated giant cells showed positive cytoplasmic staining, mainly in two of the seven patients' samples (FIG. 2C, I). Alveolar macrophages were negative or exhibited only a very weak staining (FIG. 2C, II), whereas eosinophils and lymphocytes were negative.

TABLE 2
BALF Cell Characteristics

| | Total | Analyzed | Cell Population (%)*** | | | | | |
|---|---|---|---|---|---|---|---|---|
| | BALF | BALF | | | Alveolar | Giant | | |
| | Cells | Vol. (ml) | PMN | Lymphocytes | Macrophages | Cells | Eosinophils | Unidentified |
| 1 | 4 × 10⁶ | 18 | 76** | 5 | 10 | 3 | 2 | 4 |
| 2 | 15 × 10⁶ | 15* | 92 | 1 | 3 | 1 | — | 3 |
| 3 | 14 × 10⁶ | 20* | 88** | 2 | 4 | 3 | 1 | 2 |
| 4 | 8 × 10⁶ | 20* | 80 | 3 | 3 | 9 | 1 | 4 |
| 5 | 6 × 10⁶ | 20* | 70 | 5 | 15 | 5 | 1 | 4 |
| 6 | 9 × 10⁶ | 25* | 81** | 2 | 6 | 7 | — | 4 |
| 7 | 11 × 10⁶ | 25* | 84** | 3 | 5 | 4 | 1 | 3 |

*Double gradient centrifugation was also performed to obtain pure PMN population for qRT-PCR
**FACS analysis was available in these BALF.
***Platelets were not found in BALF

Although peripheral blood neutrophils express a low amount of TF, the TF expression in BALF neutrophils was notably higher. This observation was corroborated by flow cytometry analysis (FIG. 2B). These data strongly suggest that neutrophil stimulation occurs locally within the lumen of the pulmonary alveoli or the lung micro-circulation.

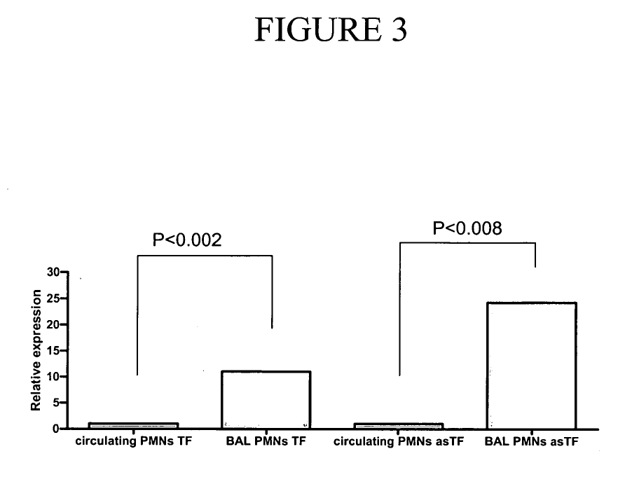

To confirm that the TF visualized in BALF neutrophils by immunohistochemical staining and flow cytometry was indeed produced by these cells, real-time PCR analysis was performed using RNA extracted from purified BALF neutrophils (purity more than 95%, see materials and methods, preparation of BALF section), and patients' peripheral blood neutrophils. This analysis showed that expression of TF mRNA was significantly higher in BALF neutrophils than in peripheral blood neutrophils from the same patients, with the alternative splicing form of TF (asTF) being the dominant isoform (FIG. 3 and Table 1).

### EXPERIMENTAL EXAMPLE 3

### Mediators Present in Alveolar Fluid in Patients with ARDS Induce the Expression of TF in Neutrophils

The up-regulation of functionally active TF in alveolar neutrophils and the lack of such up-regulation in peripheral blood neutrophils suggest that these cells are stimulated to produce TF locally in the lumen of alveoli, or when they are crossing endothelial barrier or are present in the lung microcirculation. To test the first possibility, freshly isolated neutrophils from healthy donors were incubated with 40 μl of BALF supernatant from ARDS patients. This dose was selected because, as shown by our previous experiments, this amount of BALF did not result in a shortening of the mPT. Therefore, the residual amount of TF originating from the BALF used for neutrophil stimulation was not expected to interfere with the mPT assay, which used supernatants from activated neutrophils after stimulation. Indeed, supernatants isolated from BALF-activated neutrophils were observed to exert procoagulant properties inducing a significant shortening (p<0.001) of the mPT to 24.13±1.31 sec (FIG. 1, bar 5) when compared to controls (FIG. 1, bars 1 and 2). This pro-coagulant activity was TF-dependent, since addition of anti-TF antibody to the supernatants from the activated neutrophils abrogated the pro-coagulant effect (31.73±0.37 sec, FIG. 1, bar 6).

Conversely, sera from ARDS patients did not induce significant pro-coagulant activity in neutrophils isolated from healthy individuals (29.69±1.64 sec, FIG. 1, bar 7). These results suggest that mediators that can induce the expression of functionally active TF in neutrophils are present in the alveolar fluid of ARDS patients.

### EXPERIMENTAL EXAMPLE 4

### C5a and TNF-α Stimulate Alveolar Neutrophils to Produce TF

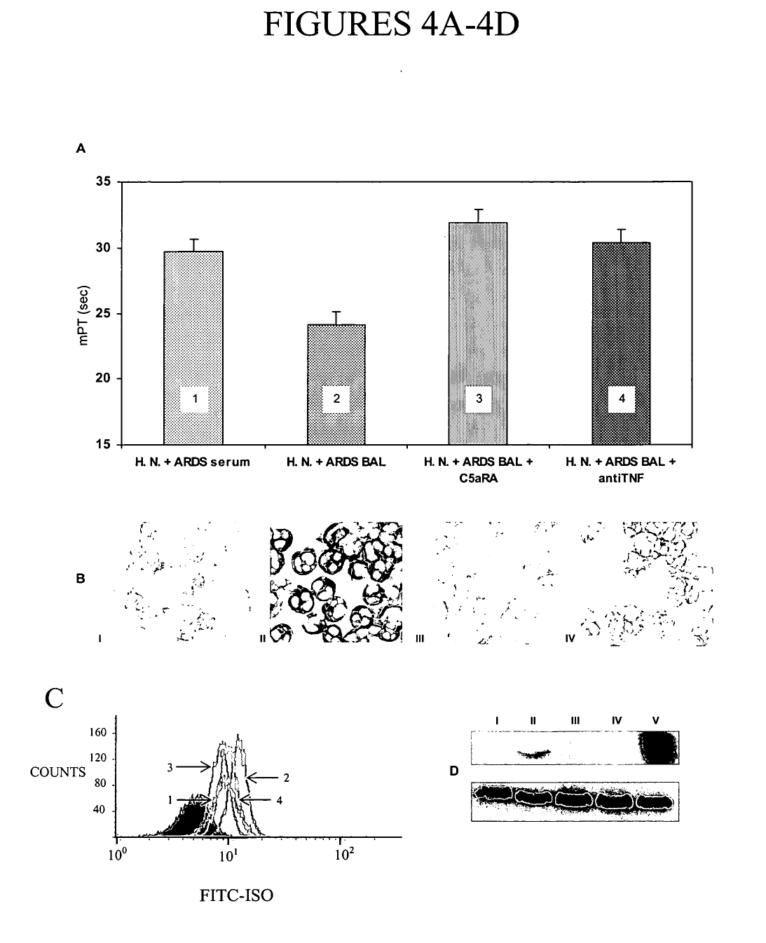

Inflammatory mediators, including complement anaphylatoxins and cytokines, enhance the thrombogenicity of blood by up-regulating TF in circulating leukocytes and endothelial cells (29). As shown in FIG. 4E, the inventors confirmed that C5a, TNF-alpha and IL-6 are indeed detectable in BALF from ARDS patients. It was therefore hypothesized that complement anaphylatoxins or/and cytokines present in alveolar exudate might stimulate neutrophils to produce TF in the course of ARDS.

To test this hypothesis, the pro-coagulant activity of supernatants from healthy volunteers' neutrophils stimulated by BALF from ARDS patients was determined, before and after treatment of the neutrophils to produce pharmacological blockade of C3a, C5a, TNF-α, or IL-6. The mPT analysis showed that the blockade of anaphylatoxin C5a or cytokine TNF-α signaling inhibited the BALF-induced pro-coagulant activity of neutrophil supernatants and resulted in significant increase (p<0.001) of the mPT values to 31.91±0.64 sec (FIG. 4A, bar 3) and 30.43±0.52 sec (FIG. 4A, bar 4) respectively, when compared to the mPT of healthy neutrophils incubated with BALF (24.13±1.31 sec, FIG. 4A, bar 2). Thus, the blockade of C5a or TNF-α signaling returned the mPT to values for control cells stimulated with ARDS serum (29.69±1.64 sec, FIG. 4A, bar 1). In contrast, a lack of C3a or IL-6 signaling did not influence the mPT values of supernatants from BALF-stimulated neutrophils.

The loss of BALF-induced pro-coagulant activity as a result of the blockade of C5a or TNF-α signaling was associated with the loss of TF expression, as demonstrated by immunohistochemical, flow cytometry and western blot analyses (FIGS. 4B, C and D). The observation that inhibition of TNF-α signaling inhibits TF expression in neutrophils demonstrates that the role of TNF-alpha in ARDS pathophysiology extends beyond the recruitment of neutrophils to ARDS-affected lungs, suggested in other work (44).

### EXPERIMENTAL EXAMPLE 5

### BALF from ARDS Patients Contains Numerous Chemokines Facilitating Migration of Neutrophils into Pulmonary Alveoli

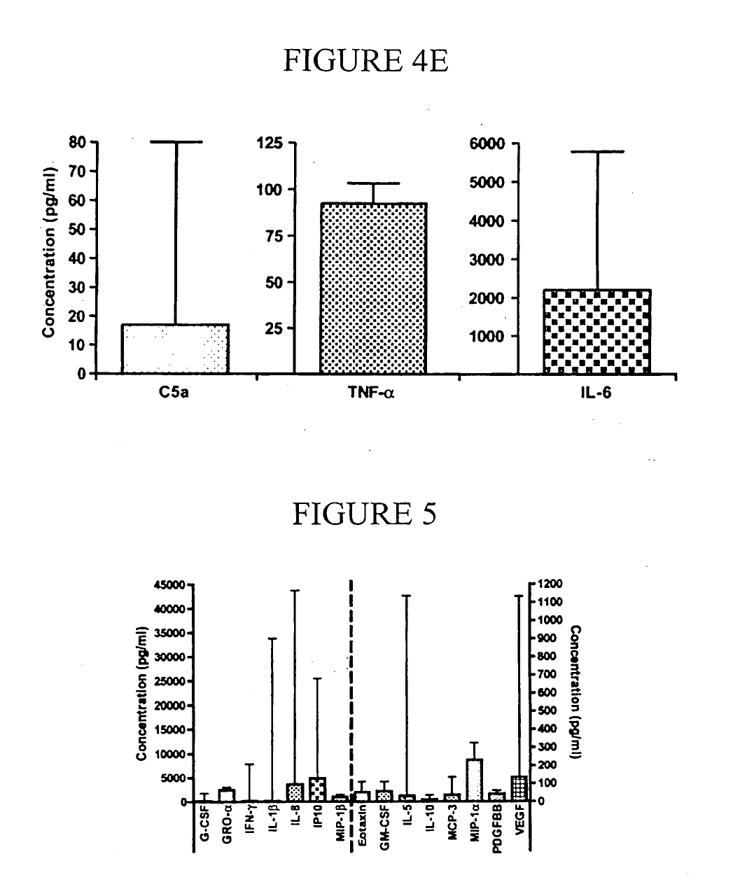

To further characterize the inflammatory microenvironment in pulmonary alveoli of ARDS-affected lungs, an assessment of levels of various cytokines, growth factors and inflammatory mediators in ARDS BALF was conducted. The concentrations of factors that were detectable in at least four out of seven BALF samples are shown in FIG. 5. Notably, various chemokines that are known to facilitate migration of neutrophils into inflamed tissues or to be expressed by neutrophils, including IL-8, GRO alpha, MIP-1 alpha, MIP-1 beta, and IP-10, were present in ARDS BALF in high concentrations. In addition, several mediators that induce the expression of chemokines in neutrophils were also detectable in ARDS BALF, including TNF-alpha (see FIG. 4), IL-1 beta, and GM-CSF (see FIG. 5).

Thus, in addition to chemokines that were already known to be present in the intra-alveolar environment of ARDS affected lungs, it is demonstrated herein that BALF contains various other mediators not previously reported as being associated with ARDS pathology. Several of these chemokines can be produced by neutrophils themselves and further amplify accumulation of these cells in inflamed tissues (44).

### EXPERIMENTAL EXAMPLE 6

### Inhibition of C3 Cleavage Reduces Procoagulation Activity of BAL Fluid from ARDS Patients

In the complement cascade, cleavage of C3 produces C3a and C3b. C3b contributes to the formation of enzymatic assemblies that in turn catalyze the formation of C5a from C5. The following experiment was designed to test whether inhibition of C3 cleavage, which reduces the amount of C5a formed, is effective in reducing TF expression. Modified PT assays were carried out essentially as described above using neutrophils obtained from healthy individuals and incubating them with BAL fluid from patients with ARDS. The BAL fluid from the seven patients was tested individually.

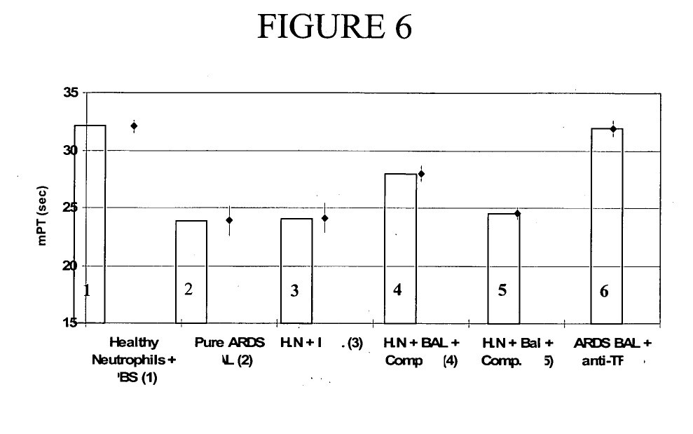

The results are summarized in FIG. 6.

Pure ARDS BAL fluid (FIG. 6, column 2, mPT=23.91±1.30) has significantly higher procoagulant activity compared to supernatants of healthy neutrophils (H.N) incubated with PBS (FIG. 6, column 1, mPT=32.11±0.58, p<0.001), which is TF-dependent (FIG. 6, column 6, mPT=31.95). H.N incubated with BAL fluid (1:5) from patients with ARDS (FIG. 6, column 3, mPT=24.13±1.31, p<0.001 compared to column 1) demonstrated increased procoagulant activity as well. In 4 cases (group a) this procoagulant activity was abolished when BALF was preincubated with compstatin (FIG. 6, column 4, mPT=27.98±0.71, p<0.001 compared to column 3). In contrast with 3 other cases (group b), incubation of BAL fluid with compstatin did not affect its procoagulant activity on healthy neutrophils (FIG. 6, column 5, mPT=24.57±0.55, P=ns when compared to column 3).

Thus, it is evident that in at least some cases of ARDS, inhibition of C3 cleavage reduces the procoagulant activity of BAL fluid.

### REFERENCES

1. Frutos-Vivar F, Nin N, Esteban A: Epidemiology of acute lung injury and acute respiratory distress syndrome. Curr Opin Crit Care 2004; 10:1-6
2. Ventilation with lower tidal volumes as compared with traditional tidal volumes for acute lung injury and the acute respiratory distress syndrome. The Acute Respiratory Distress Syndrome Network. N Engl J Med 2000; 342:1301-1308
3. Idell S: Extravascular coagulation and fibrin deposition in acute lung injury. New Horiz 1994; 2:566-574
4. Nucton T J, Alonso J A, Kallet R H, et al. Pulmonary dead-space fraction as a risk factor for death in the acute respiratory distress syndrome. N Engl J Med 2002; 346:1281-1286
5. Idell S, James K K, Levin E G, et al: Local abnormalities in coagulation and fibrinolytic pathways predispose to alveolar fibrin deposition in the adult respiratory distress syndrome. J Clin Invest 1989; 84:695-705
6. Gunther A, Mosavi P, Heinemann S, et al: Alveolar fibrin formation caused by enhanced procoagulant and depressed fibrinolytic capacities in severe pneumonia. Comparison with the acute respiratory distress syndrome. Am J Respir Crit Care Med 2000; 161:454-462
7. Fuchs-Buder T, de Moerloose P, Ricou B, et al: Time course of procoagulant activity and D dimer in bronchoalveolar fluid of patients at risk for or with acute respiratory distress syndrome. Am J Respir Crit Care Med 1996; 153:163-167
8. Idell S, Koenig K B, Fair D S, et al: Serial abnormalities of fibrin turnover in evolving adult respiratory distress syndrome. Am J Physiol 1991; 261:L240-L248
9. Giesen P, Rauch U, Bohrmann B, et al: Blood-borne tissue factor: another view of thrombosis. Proc Natl Acad Sci USA 1999; 96:2311-2315
10. Osterud B: Tissue factor expression by monocytes: regulation and pathophysiological roles. Blood Coagul Fibrinolysis 1998; 1(Suppl):S9-S14
11. Szotowski B, Antoniak S, Poller W, et al: Procoagulant soluble tissue factor is released from endothelial cells in response to inflammatory cytokines. Circ Res 2005; 96:1233-1239
12. Bachofen M, Weibel E R: Structural alterations of lung parenchyma in the adult respiratory distress syndrome. Clin Chest Med 1982; 3:35-56
13. Meyrik B: Pathology of the adult respiratory distress syndrome. Crit Care Clin 1986; 2:405-429
14. Dunnill M S: Shock lung and the adult respiratory distress syndrome. In: Dunnill M L, ed. Pulmonary Pathology. 2^{nd} ed. New York, N.Y.: Churchill Livingstone, 1987; 31-40
15. Kinoshita M, Mochizuki H, Ono S: Pulmonary neutrophil accumulation following human endotoxemia. Chest 1999; 116:1709-1715
16. Sakamaki F, Ishizika A, Urano T, et al: Effect of a specific neutrophil elastase inhibitor, ONO-5046, on endotoxin-induced acute lung injury. AM J Respir Cric Care Med 1996; 153:391-397
17. Cirelli R A, Carey L A, Fisher J K, et al: Endotoxin infusion in anesthetized sheep associated with intrapulmonary sequestration of leukocytes that immunohistochemically express tumor necrosis factor-a. J Leukoc Biol 1995; 57:820-826
18. Ritis K, Doumas M, Mastellos D, et al: A novel C5a receptor-tissue factor cross-talk in neutrophils links innate immunity to coagulation pathways. J Immunol 2006; 177:4794-4802
19. Huber-Lang M, Sarma J V, Zetoune F S, et al: Generation of C5a in the absence of C3: a new complement activation pathway. Nat Med 2006; 12:682-687
20. Bernard G, Artigas A, Brigham K, et al: The American-European Consensus Conference on ARDS. Am J Respir Crit Care Med 1994; 149:818-824
21. Nakos G, Pneumatikos J, Tsangaris I, et al: Proteins and phospholipids in BAL from patients with hydrostatic pulmonary edema. Am J Respir Crit Care Med 1997; 155(3):945-951
22. Ames R, Lee D, Foley J, et al: Identification of a selective nonpeptide antagonist of the anaphylatoxin C3a receptor that demonstrates anti-inflammatory activity in animal models. J Immunol 2001; 166:6341-6348
23. Mastellos D, Papadimitriou J, Franchini S, et al: A novel role of complement: mice deficient in the fifth component of complement (C5) exhibit impaired liver regeneration. J Immunol 2001; 166:2479-2488
24. Lappegard K, Riesenfeld J, Brekke O, et al: Differential effect of heparin coating and complement inhibition on artificial surface-induced eicosanoid production. Ann Thorac Surg 2005; 79:917-923
25. Bogdanov V, Balasubramanian V, Hathcock J, et al: Alternatively spliced human tissue factor: a circulation soluble, thrombogenic protein. Nat Med 2003; 458-462
26. Szotowski B, Goldin-Lang P, Antoniak S, et al: Alteration in myocardial tissue factor expression and cellular localization in dilated cardiomyopathy. J Am Coll Cardiol 2005; 45:1081-1089
27. Livak K, Schmittgen T: Analysis of relative gene expression data using real time quantitative PCR and the 2^{-2ΔΔC}_{T} method. Methods 2001; 25:402-408
28. Cordell J, Fallini B, Erber W, et al: Immunoenzymatic labelling of monoclonal antibodies using immune complexes of alkaline phosphatise and monoclonal antialkaline phosphatase (APAAP complexes). J Histochem Cytochem 1984; 32:219-229
29. Markiewski M M, Nilsson B, Nilsson Ekdahl K, et al: Complement and coagulation: strangers or partners in crime? Trends Immunol 2007; 28(4):184-192
30. Reutershan J, Ley K: Bench-to-bedside review: Acute respiratory distress syndrome—how neutrophils migrate into the lung. Crit Care 2004; 8:453-461
31. Ognibene F P, Martin S E, Parker M M, et al: Adult respiratory distress syndrome in patients with severe neutropenia. N Engl J Med 1986; 315:547-551
32. Azoulay E, Darmon M, Delclaux C, et al: Deterioration of previous acute lung injury during neutropenia recovery. Crit Care Med 2002; 30:781-786
33. Doerschuk C M, Quinlan W M, Doyle N A, et al: The role of P-selectin and ICAM-1 in acute lung injury as determined using blocking antibodies and mutant mice. J Immunol 1996; 157:4609-4614
34. Kamochi M, Kamochi F, Yim Y B, et al: P-selectin and ICAM-1 mediate endotoxin-induced neutrophil recruitment and injury to the lung and liver. Am J Physiol Lung Cell Mol Physiol 1999; 277:L310-L319
35. Heflin A C Jr, Brighman K L: Prevention by granulocyte depletion of increased vascular permeability of sheep lung following endotoxemia. J Clin Invest 1981; 68:1253-1260
36. Till G O, Johnson K T, Kunkel R, et al: Intravascular activation of complement and acute lung injury. Dependency on neutrophils and toxic oxygen intermediates. J Clin Invest 1982; 69:1126-1135
37. Abraham E, Carmody A, Shenkar R, et al: Neutrophils as early immunologic effectors in hemorrhage- or endotoxemia-induced acute lung injury. Am J Physiol Lung Cell Mol Physiol 2000; 279:L1137-1145
38. Mulligan M S, Schmid E, Beck-Schimer B, et al: Requirement and role of C5a in acute lung inflammatory injury in rats. J Clin Invest 1996; 98:503-512
39. Harkin D W, Marron C D, Rother R P, et al: C5 complement inhibition attenuates shock and acute lung injury in an experimental model of ruptured abdominal aortic aneurysm. Br J Surg 2005; 92:1227-1234
40. Suter P M, Suter S, Girardin E, et al: High Bronchoalveolar levels of tumor necrosis factor and its inhibitors, interleukin-1, interferon, and elastase, in patients with adult respiratory distress syndrome after trauma, shock, or sepsis. Am Rev Respir Dis 1992; 145:1016-1022
41. Hyers T M, Tricomi S M, Dettenmeier P A, et al: Tumor necrosis factor levels in serum and bronchoalveolar lavage fluid of patients with the adult respiratory distress syndrome. Am Rev Respir Dis 1991; 144:268-271
42. Meduri G U, Kohler G, Headley S, et al: Inflammatory cytokines in the BAL of patients with ARDS. Persistent elevation over time predicts poor outcome. Chest 1995; 108:1303-1314
43. Yoshinari D, Takeyoshi I, Koibuchi Y, et al: Effects of a dual inhibitor of tumor necrosis factor-α and interleukin-1 on lipopolysaccharide-induced lung injury in rats: Involvement of the p38 mitogen-activated protein kinase pathway. Crit Care Med 2001; 29: 628-634
44. Schnyder-Candrian S, Quesniaux V, Padova F, et al: Dual effects of p38 MAPK on TNF-dependent bronchoconstriction and TNF-independent neutrophil recruitment in lipopolysaccharide-induced acute respiratory distress syndrome. J Immunol 2005; 175: 262-269
45. Oikonomou N, Harokopos V, Zalevsky J, et al: Soluble TNF mediates the transition from pulmonary inflammation to fibrosis. PLoS ONE 2006; 1:e108.doi:10.1371/journal.pone.0000108
46. Azoulay E, Herigault S, Levame M, et al: Effect of granulocyte colony-stimulating factor on bleomycin-induced acute lung injury and pulmonary fibrosis. Crit Care Med 2003; 31:1442-1448
47. Guery B P, Welsh D A, Viget N B, et al: Ventilation induced lung injury is associated with an increase in gut permeability. Shock 2003; 19:559-563

The present invention is not limited to the embodiments described and exemplified herein, but is capable of variation and modification within the scope of the appended claims.

What is claimed:

1. A method for treating acute respiratory distress syndrome (ARDS) in an individual, the method comprising administering a therapeutically effective amount of a complement inhibitor or a TNF-alpha inhibitor to the individual, wherein the complement inhibitor or TNF-alpha inhibitor reduces or prevents tissue factor production in alveolar neutrophils, thereby treating the ARDS.

2. The method of claim 1, wherein the complement inhibitor comprises one or more of a C5a inhibitor, a C5aR inhibitor, a C3 inhibitor, a Factor D inhibitor, a Factor B inhibitor, or a combination thereof.

3. The method of claim 2, wherein the complement inhibitor is a C5a inhibitor or a C5aR inhibitor.

4. The method of claim 3, wherein the C5a inhibitor or C5aR inhibitor is acetyl-Phe-[Orn-Pro-D-cyclohexylalanine-Trp-Arg] (PMX-53), PMX-53 analogs, neutrazumab, TNX-558, eculizumab, pexelizumab or ARC 1905, or any combination thereof.

5. The method of claim 2, wherein the complement inhibitor is a C3 inhibitor.

6. The method of claim 5, wherein the C3 inhibitor is compstatin, a compstatin analog, a compstatin peptidomimetic, a compstatin derivative, or any combinations thereof.

7. The method of claim 1, wherein the TNF-alpha inhibitor is an antibody that binds and inhibit TNF-alpha activity, or a soluble TNF-alpha receptor, or a tyrosine kinase inhibitor.

8. The method of claim 1, wherein the tissue factor is alternatively spliced tissue factor isoform.

9. The method of claim 1 wherein the individual is human.

10. The method of claim 1, wherein the reducing or preventing of tissue factor production in alveolar neutrophils results in reduction or prevention of intra-alveolar fibrin deposition.

11. The method of claim 1, wherein the complement inhibitor is administered by pulmonary administration.

12. A method for treating an individual at risk for developing acute respiratory distress syndrome (ARDS), the method comprising administering a therapeutically effective amount of a complement inhibitor or a TNF-alpha inhibitor to the individual, wherein the complement inhibitor or TNF-alpha inhibitor reduces or prevents tissue factor production in alveolar neutrophils, thereby treating the ARDS.

13. The method of claim 12, wherein the complement inhibitor comprises one or more of a C5a inhibitor, a C5aR inhibitor, a C3 inhibitor, a Factor D inhibitor, a Factor B inhibitor, or a combination thereof.

14. The method of claim 13, wherein the complement inhibitor is a C5a inhibitor or a C5aR inhibitor.

15. The method of claim 14, wherein the C5a inhibitor or C5aR inhibitor is acetyl-Phe-[Orn-Pro-D-cyclohexylalanine-Trp-Arg] (PMX-53), PMX-53 analogs, neutrazumab, TNX-558, eculizumab, pexelizumab or ARC 1905, or any combination thereof.

16. The method of claim 12, wherein the complement inhibitor is a C3 inhibitor.

17. The method of claim 16, wherein the C3 inhibitor is compstatin, a compstatin analog, a compstatin peptidomimetic, a compstatin derivative, or any combinations thereof.

18. The method of claim 12, wherein the TNF-alpha inhibitor is an antibody that binds and inhibit TNF-alpha activity, or a soluble TNF-alpha receptor, or a tyrosine kinase inhibitor.

19. The method of claim 12, wherein the tissue factor is alternatively spliced tissue factor isoform.

20. The method of claim 12 wherein the individual is human.

21. The method of claim 12, wherein the reducing or preventing of tissue factor production in alveolar neutrophils results in reduction or prevention of intra-alveolar fibrin deposition.

22. The method of claim 12, wherein the complement inhibitor is administered by pulmonary administration.

23. The method of claim 12, wherein the individual at risk of developing ARDS exhibits one or more clinical conditions of acute lung injury (ALI), sepsis and the systemic inflammatory response syndrome (SIRS), severe traumatic injury, severe head injury, pulmonary contusion; severe pulmonary infection, aspirated vomited stomach contents, inflamed pancreas, severe trauma with shock, multiple transfusions, near drowning, smoke inhalation, or overdoses of narcotics, salicylates, tricyclic antidepressants or other sedatives.


SEQUENCE LISTING (3 sequences)

SEQ ID NO: 1 - protein, 13 aa, Artificial sequence
  synthetic sequence
ICVVQDWGHHRCT

SEQ ID NO: 2 - protein, 16 aa, Artificial Sequence
  synthetic sequence
XXCVXQDWGXHRCXXX

SEQ ID NO: 3 - protein, 16 aa, Artificial Sequence
  synthetic sequence
XXCVXQDXGXHRCXXX